(19) United States
(12) Patent Application Publication
(10) Pub. No.: US 2021/0346717 A1
(43) Pub. Date: Nov. 11, 2021

(54) Rigel

(71) Applicant: Rosirlene Galo Young, Seattle, WA (US)

(72) Inventor: Rosirlene Galo Young, Seattle, WA (US)

(21) Appl. No.: 17/329,743

(22) Filed: May 25, 2021

Related U.S. Application Data
(60) Provisional application No. 63/037,352, filed on Jun. 10, 2020.

(51) Int. Cl. A61N 5/06; G06F 1/16

(52) U.S. Cl. CPC A61N 5/0624; A61N 5/0616; G06F 1/163; A61N 2005/0653; A61N 2005/0652; A61N 2005/0645; A61N 2005/0663; G06F 1/1652

(57) ABSTRACT

The present invention relates to a wearable smart device that controls, inhibit, and abates bacteria, virus, and fungi; The invention provides method and device to sanitize areas, spaces, surfaces, and items as well as human skin: The invention can be selectively used by an individual as desired to assist in control and abate the individual's hands, feet, footwear, or area: The invention is comprised of a kind of wearable smart device that forms a loop which can be placed around an individual's wrist or ankles, an off and on small light that serves as a switch at the upper side of the device and a universal base charging port by the side of the device used in charging the unit or the device can be stationary in one location and plug to the power source.

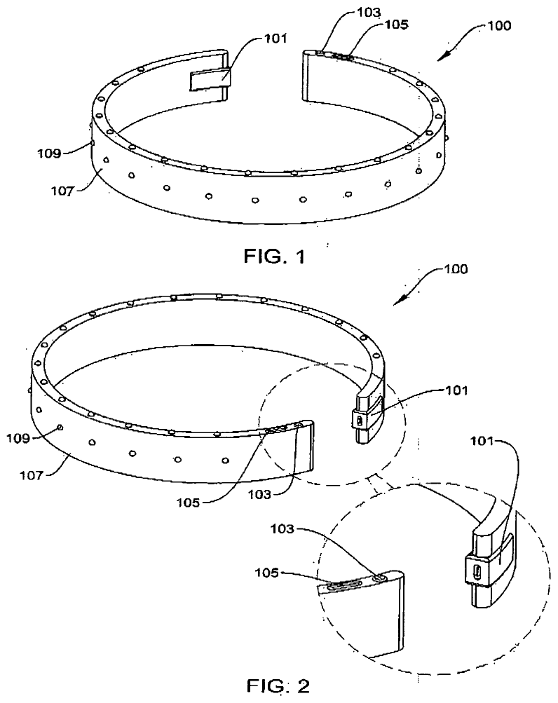

Device specifically adapted for protection against bacteria, virus and fungi that attacks us from our environment and interaction with people; It uses light therapy, a very researched and studied therapy; It is a product related to the accessories, technology, and that health sector;

### BACKGROUND

### Field of the Invention

The method and device object are to provide a method and the device that controls, inhibit, and abates bacteria, viruses, fungi from skin, areas, spaces, surfaces, it also prevents premature aging of skin; It is a product related to the accessories, technology and health sector, It provides more practicality, safety, free of chemicals and toxins to the user in his/her daily activities, It is an object to provide a device and a method of control and abates bacteria, viruses, fungi by luminescence with one or more LED elements emitting light on a wavelength within a range of 749 to 959 nm, and one or more LED elements emitting light a wavelength within a range of 100 to 227 nm, one or more LED elements emitting light on a wavelength within a range of 379 to 474 nm, one or more LED elements emitting light on a wavelength within a range of 474 to 701 nm, it is a further object of the invention to provide a method for control and abates by luminescence with one or more LED or OLED elements, plurality of light emitting diodes (LEDs), It is an object of this invention to provide a device for personal hygiene use in public facilities and areas, a device that control and abates bacteria, viruses, fungi preventing contamination, cross-contamination; and it also stimulates skin repair to preventing premature aging of skin, It is an object of this invention to provide a device capable of controls, inhibit, and abates bacteria, viruses, fungi within one second to sixty seconds,

A method and device comprising of a unit base shape, which is to be worn on the wrist or ankles and it possess multi lights over an inner side of the device and surface or can be stationary in one location and plug to the power source; It's a smart device that has a compatibility for apps, can collect, store and transmit data and vital data;

### DETAILS OF RELATED INVENTION

Photomatrix Device U.S. Pat. No. 6,443,978

A device for the physiotherapeutic irradiation of spatially extensive pathologies by light with the help of a matrix of the sources of optical radiation such as lasers or light diodes placed on the surface of a substrate whose shape is adequate to the shape of the zone of pathology is disclosed; In addition, the device contains stops and a holder to fix the substrate against the bioobject; Additional modules are provided to adjust the temperature, pressure, gas composition over the pathological area; As a source of radiation, chemical reactions accompanied by the luminescence of the products of reaction are suggested; The power supply unit can be autonomous with remote feeding through a pulse magnetic field; A supplementary hood optically transparent is provided to localize the pathology as well as the strips that scatter the radiation to get a more uniform bioobject's exposure; Application: light-therapy to treat various extensive pathologies on the bioobject's surface including dermatology, cosmetology; the treatment of traumas, bruises, oedemas, varicose veins, blood therapy, treatment of infectious processes;

Antiseptic Bracelet—U.S. Pat. No. 9,578,935

A wearable device containing an antiseptic solution that can be selectively dispensed by a user as desired to assist in disinfecting the user's hands or other surface; The antiseptic bracelet is to be worn on and around user's wrist near the base of the user's palm and comprises a device band that is connected to opposing sides of a dispensing pod, forming a loop which can be placed around a user's wrist; The dispensing pod includes a hollow interior forming an internal reservoir and a dispensing tip positioned in an outlet channel; The internal reservoir enables the dispensing pod to bold an antiseptic solution and dispensing tip enables a user to selectively dispense a portion of the solution in the dispensing pod;

Airborne Pathogen Neutralization Canada (2459041)

A method and apparatus is described for neutralizing airborne pathogens in ventilated air, and in heating or air conditioning systems; The pathogen neutralization system is effective against a wide spectrum of pathogens, it incorporates commercially available components, and it can be readily integrated into commercial HVAC systems where it neutralizes airborne pathogens in large volumes of ventilated air in real time without any chemical reagents; Typically, the system has a flow-through reaction chamber that contains a UV light source that emits short intense flashes of broad-spectrum UV light, a source of water vapor or spray, and an ozone generator; The system generates highly reactive ozone intermediates by irradiating ozone gas with UV light in the presence of water droplets or water vapor; The pathogens that can be neutralized by this system include bacteria, viruses, spores, fungi and parasites;

Device for facial treatment based on light emission and induced magnetic field, and system including same; U.S. Pat. No. 10,105,548 The present invention provides a facial treatment device and a system including the treatment device combining light and electromagnetic therapy for facial treatments; The facial treatment device includes a mask base; a plurality of light emitting diodes (LEDs) disposed across an inner side of the facial treatment device; and one or more electromagnetic field producing units, each configured for producing an electromagnetic field located over the periphery of the mask base; The system includes the facial treatment device and a control unit that allows controlling the operation of the LEDs and the one or more electromagnetic field producing units;

Compositions and methods of invasive and now-invasive procedural skincare, U.S. Pat. No. 10,286,030 Compositions and methods for skincare treatment are provided; The invention relates more generally to skin care treatment and more particularly, to compositions and methods for promoting healthy skin, skin regeneration, skin repair, skin bed preparation, and enhanced wound healing;

Disinfecting lighting device U.S. Pat. No. 10,357,582 Disclosed herein is a device which inactivates microorganisms; The device includes a light emitter and at least one light-converting material arranged to convert at least a portion of light from the light emitter; Any light emitted from the light emitter and converted light emitted from at least one light-converting material mixes to form a combined light, the combined light having a proportion of spectral energy measured in an approximately 380 nm to approximately 420 nm range of greater than approximately 10 percent; In another embodiment, the device includes a light emitter configured to emit light with wavelengths in a range of 380 to 420 nm, and at least one light-converting material including at least one optical brightener and configured to emit a second light; The first light exiting the device and the second light exiting the device mix to form a combined light, the combined light being white;

Portable light fastening assemble; U.S. Pat. No. 10,413,624 A portable light fastening assembly for use with the human interface of an electronic device includes a lamp housing and an adjustable attachment device extending from the lamp housing; An ultra-violet (UV) light source is at least partially enclosed in the lamp housing such that the adjustable attachment device includes an engagement member and a receptacle housing such that the engagement member can be removably fastened within the receptacle housing for holding the lamp housing in a fixed position;

Light Therapy spot applicator; U.S. Pat. No. 10,518,105 LED lights systems comprising of a therapeutic lamp platform for radiant lamps such as LEDs disposed in a holdable spot applicator assembly, the holdable spot applicator assembly including a reflective surface facing towards a patient and a plurality of LEDs for communicating lamp radiation from the lamps to a user; The lamps and associated circuitry are housed within a holdable elongated structure;

Fingernail dryer; U.S. D878,68 The ornamental design for a fingernail dryer,

Cover with disinfecting illuminated surface, U.S. Pat. No. 10,617,774 A light emitting cover which inactivates microorganisms for covering a high touch surface is disclosed; The light emitting cover includes a body having an interior configured to cover at least a portion of the high touch surface and an exterior surface configured to be disinfected; At least an exterior portion of the body is transparent or translucent; A light emitter operably is coupled to the body for emitting a light through the exterior surface; The light exiting the exterior surface has at least a portion thereof having a wavelength in a range of 380 to 420 nanometers to disinfect the exterior surface;

Movable ultraviolet radiation beam; U.S. Pat. No. 10,588,33 A solution for irradiating a surface with ultraviolet radiation is provided; A movable optical element is utilized to form a beam of ultraviolet radiation having a characteristic cross-sectional area smaller than an area of the surface to be irradiated; The movable optical element can be moved as necessary to directly irradiate any portion of the surface with radiation within the characteristic cross-sectional area of the beam of ultraviolet radiation; The movement can include, for example, rotational movement and/or repositioning the movable ultraviolet source with respect to the surface;

Sterilization device utilizing low intensity UV-C radiation and ozone; U.S. Pat. No. 10,596,282 A sterilization system uses ultraviolet radiation and ozone to eradicate deadly pathogens to sterilize a zone of a surface of an object or wound/incision sites; The sterilization system offers a safe sterilization method prior to or after surgery and/or closure wounds/incisions; The sterilization system has ultraviolet emitters that emit ultraviolet light in wavelengths that kill pathogens and in wavelengths that produce ozone at the sites, therefore killing/disabling several hard-to-kill pathogens; The sterilization system has proximity detectors to enable ultraviolet emission only when the system is properly located near or against the surface so as to protect from unwanted radiation from the ultraviolet light;

Photoeradication of microorganisms with pulsed purple or blue light; U.S. Pat. No. 10,639,498 The present invention is directed to a system and method for photoeradication of microorganisms from a target; A light source is configured to irradiate the target with purple or blue light in a pulsed mode of irradiation at an average irradiance that preferably ranges from 0.1 mW/cm2 to 20 mW/cm2; An electronic circuit is configured to control the light source in accordance with an irradiation schedule that includes a plurality of irradiation sessions; The light source is controlled to provide the purple or blue light at a radiant exposure that preferably ranges from 0.5 J/cm2 to 60 J/cm2 during each of the irradiation sessions; The irradiation sessions are provided at a plurality of time intervals so as to cause photoeradication of all or a portion of the microorganisms;

### DETAILED DESCRIPTION

It is well known that the number one form of infectious disease is spread is through hand contact or cross-contamination of footwear; This is why the United States Center for Disease Control and governmental bodies all over the world recommend regular hand washing and the use of germicidal solutions and properly cleaned and sanitized footwear; One problem with such a recommendation, however, is the fact that clean water and soap or germicidal solutions are not always immediately available to those who need to clean their hands at the time and place of need; Same for the footwear, indeed, the creation of a germicidal device in the first instance was in large part to address this problem as people are able to transport and or keep a germicidal device worn on their wrist or ankles for constant availability with much greater ease than water and soap;

While the proliferation of germicidal solutions helped address the problem, it did not completely solve it; A problem that still exists because the use of germicidal solutions as presently implemented typically require a user to carry around a bottle containing such a solution or be in a close proximity to a germicidal solution or a wall mount or other stationary location; furthermore, it is understood that one issue which arises with carrying and pulling out a bottle of germicidal solution can often lead to contamination of the surface of the bottle as it would typically require a full hand to pull out, open, dispense, and put away; In addition, in some social settings, it is frowned upon to pull out bottled germicidal solutions after meeting an individual or group and shaking hands; Also FDA warns of methanol-tainted hand sanitizer—but can't force companies to recall it; Seventeen people have died in 2020 after being exposed to hand sanitizer made with methanol, a highly toxic chemical, the FDA says;

Glove Corporation—the world's largest producer of gloves by volume—is expecting profits to jump by up to 40% in its fiscal second half of 2020 due to a surge in demand amid the COVID-19 global pandemic; By December 2021, Top Glove is projected to have a total of 861 production lines and a capacity of 91 billion gloves per year; French nonprofit Opération Mer Propre, or Operation Clean Sea, warns ‘COVID waste’ could harm the environment by trigging a new wave of plastic pollution; Gloves are unrecyclable and millions end up in our landfills every year and our oceans; These disposable gloves can take decades to break down; Even gloves made from latex (a natural rubber) take a long time to biodegrade because other chemicals added to the gloves; The need for a replacement of the gloves and latex gloves are a must with something that is free of chemicals and toxins and safe for the environment;

The benefits of light therapy also known as phototherapy is a well-documented research and has been used worldwide for nearly a century; in Sep. 17, 2019, Little Sparrows Technologies, a neonatal medical device startup committed to promoting the well-being of newborns, announced it has received 510(k) clearance from the Food and Drug Administration (FDA) for the bili-hut™, The device features a unique curved light emitting diode (LED) illumination canopy that delivers high-intensity blue light phototherapy, This highly portable device allows newborns to effectively undergo jaundice treatment anywhere—at home or in a hospital setting, On Dec. 14, 2020. Little Sparrows Technologies announced that the trial results show the bili-hut™ neonatal phototherapy system delivered effective high intensity phototherapy for treating newborns in the home setting eliminating the need for infant hospital readmissions; The FDA has also approved various light sources for the treatment of dermatologic skin,

Recent studies demonstrate that various wavelengths in the blue spectrum are antimicrobial against the deadly methicillin-resistant Staphylococcus aureus (MRSA), Escherichia coli, Helicobacter pylori, Listeria monocytogenes, Pseudomonas, aeruginosa, Salmonella, Acinetobacter, baumannii, Aggregatibacter actinomycetemcomitans, Propionibacterium acnes, Neisseria, gonorrhoeae, Porphyromonas gingivalis, Fusobacterium nucleatum, and others, like the recent outbreak of COVID-19, which continues to ravage communities with high death tolls and untold psychosocial and catastrophic economic consequences, is a vivid reminder of nature's capacity to defy contemporary healthcare; The pandemic calls for rapid mobilization of every potential tool, including phototherapy—one of the most effective treatments used to reduce the impact of the 1918 “Spanish influenza” pandemic; Several studies showing that phototherapy has immense potential to reduce the impact of coronavirus diseases and other infections; Now FAR-UV is safer and more effective than the existing 250 to 280 nm UVC systems; Columbia University has a 6 year study on FAR-UVC and they came to the finding that UVC, at 222 nm light or under is safe for us because it cannot even penetrate through the dead-cell layer on the surface of our skin or the tear layer on the surface of our eyes—but because bacteria and viruses are physically very small, 222 nm light does have enough penetration to efficiently kill them; Throughout the years, science has determined the effects of various ranges of wavelengths of light, but absorption has proven to be the key to cellular change; All cells are photo-receptive and can be stimulated at differing depths dependent upon wavelength;

Skin cells respond well to light therapy involving low level light due to the fact that cells reside just underneath the skin surface, making these low levels of energy able to reach the receptor sites and induce desired photochemistry to achieve beneficial results;

Consequently, Rigel is a product related to the accessories and technology and health sector; It refers to a new system model for protection against bacteria, virus and fungi that uses lights; Rigel method and device offers much more practicality and safety a way of control, inhibit, and abate bacteria, virus, and fungi preventing contamination, cross-contamination in areas like bus, METRO, restaurant table, bar counter, public bathroom, hospital, shopping in the supermarket, workstation, assembly line, etc;

### DESCRIPTION OF DRAWINGS

FIG. #1 Front of device showing lights on the surface of device

FIG. #2 Detail of device showing USAB-C charger and ON/OFF button;

### SUMMARY OF THE INVENTION

The present invention provides disinfection method comprising of a smart device shaped system to be worn on wrist or ankles, with a plurality of lights by the side of the device emitting a light therapy towards the hand or feet covering it;

The device and method are an efficiently and cost-effectively, eco-friendly in to controls, inhibit, and abate transfer of microbes, such as bacteria, virus, and fungi, seamlessly during day-to-day activities, at the same time preventing premature aging of skin.

The invention claimed is:

1. The method and device are capable to controls, inhibit, and abates bacteria, virus, and fungi preventing contamination, cross-contamination, and stimulate skin repair to prevent premature aging of skin,

2. The method and device claim #1 controls, inhibit, and abates bacteria, virus, and fungi in the skin or in object surface by luminescence with one or more LED. elements emitting light on a wavelength within a range of 749 to 959 nm, and one or more LED elements emitting light a wavelength within a range of 100 to 227 nm: one or more LED. elements emitting light on a wavelength within a range of 379 to 474 nm, one or more LED elements emitting light on a wavelength within a range of 474 to 701 nm, it is a further object of the invention to provide a method for controls, inhibit, and abates by luminescence with one or more LED or OLED elements, plurality of light emitting diodes (LEDs),

3. The method and device claim #1, #2, provides a device capable of controls, inhibit, and abates bacteria, viruses, fungi within one second to sixty seconds reducing transmission through surfaces or skin,

4. The method and device claim #1, #2,#3 can inhibit, covid-19 transmission through surfaces,

5. The method and device claim #1, #2, #3, #4 can comprising of a unit base shape, which is to be worn on the wrist or ankles or standing alone and it has multi-LED or lights over an inner side and surface, and it can be stationary in one location and plug to the power source,

6. The method and device claim #1, #2, #3, #4, #5 is non-invasive chemical free method and device that controls, inhibit, and abates virus, fungi and bacteria by illumination using different wavelength disrupting the DNA of the bacteria, virus and fungi, triggering the body's natural cell processes to accelerate rejuvenation and repair of the skin,

7. The method and device claim #1, #2, #3, #4, #5, #6 can possess a light control switch that controls the illumination,

8. The method and device claim #1, #2, #3, #4, #5, #6, #7 can have a power supply port by its side, which supplies power when plugged to electricity using a USB-C CHARGER,

9. The method and device claim #1, #2, #3, #4, #5, #6, #7, #8 is a wearable smart device and it has compatibility for apps, can collect, store, and transmit data and vital data,

10. The method and device claim #1, #2, #3, #4, #5, #6, #7, #8, #9 can have bendable screen, flexible screen or flexible display on it or around it,

11. The method and device claim #1, #2, #3, #4, #5, #6, #7, #8, #9, #10, can possesses different colors of light around it and on its surface of the device and the lights change as it covers an area.
